(19) United States
(12) Patent Application Publication
(10) Pub. No.: US 2002/0127586 A1
(43) Pub. Date: Sep. 12, 2002

(54) Method of analyzing granular composition by fluorescene analysis

(75) Inventor: Peter Paasch Mortensen, Kalundborg (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/057,431

(22) Filed: Jan. 25, 2002

Related U.S. Application Data
(60) Provisional application No. 60/266,744, filed on Feb. 6, 2001.

(30) Foreign Application Priority Data
Jan. 31, 2001 (DK) PA 2001 00157

(51) Int. Cl. C12Q 1/68; G01N 33/53; G01N 33/543

(52) U.S. Cl. 435/6; 435/7.1; 436/518

(57) ABSTRACT

The present invention relates to a method for fluorescence analysis comprising illuminating a granular composition comprising a purified biologically active compound with light capable of fluorescence excitation of a fluorescent marker comprised in the granular composition, detecting light emitted from the fluorescent marker and predicting the amount of fluorescent marker in the granular composition accessible to fluorescence excitation.

### FIELD OF THE INVENTION

The present invention relates to a method for analyzing a property of a granular composition comprising a biologically active compound by subjecting the granular composition to fluorescence analysis. The invention also relates to a method for producing a particulate product comprising subjecting the product to fluorescence analysis. Further the invention relates to a granulation and/or coating apparatus suitable for preparing a granular composition comprising a biologically active compound said apparatus comprising means for fluorescence analysis.

### BACKGROUND OF THE INVENTION

Analysis of chemical compounds in samples exploiting that some compounds (fluorophors) exhibit fluorescence when excitated with light (fluorescence analysis) are well known to the art. While fluorescent analysis has its advantages in being sensitive and accurate in well-defined samples it also has serious drawbacks. For example in complex samples the fluorescence of a fluorophor is often altered by other compounds present in the environment surrounding the fluorophor (known as quenching) making it difficult to use a fluorescence analysis quantitatively on complex and/or poorly defined samples. This applies especially in heterogeneous or solid phase samples where also scattering of the light has to be accounted for.

During recent years developments in electronics the more sophisticated methods of fluorescent imaging using camera detectors has emerged, enabling photographing of emitted light in fluorescing samples, so that two dimensional images showing the spatial distribution of fluorophors in the sample.

One example of fluorescence imaging of aleurone tissue for flour refinement using UV excitation is known from Dexter et al., Cereal Chemistry, vol. 70(1), 90-95, 1993.

Kaufman et al., Powder Technology, vol. 78(3), 239-246, 1994) have done in situ visualization of coal particle distribution in a liquid fluidised bed using fluorescence microscopy.

More general examples of imaging in analysis is found in Buydens et al., Analytical Chimica Acta, vol. 361(1-2), 161-176, 1998 who has reported on-line classification and multivariate image analysis on plastics in waste based on imaging in the Near Infrared Range of light.

Watano et al., Pharmaceutical Bulletin, vol. 48(8), 1154-1159, 2000 has reported on-line monitoring of granule growth in high shear granulation by an imaging processing system.

Watano et al. has in U.S. Pat. No. 5,497,232 reported imaging of granule growth in a fluid bed or a pan type granulator using a photographic camera such as a CCD camera of the type used in video cameras.

### SUMMARY OF THE INVENTION

The present invention relates to a method for analyzing a property of a granular composition comprising a purified biologically active compound by subjecting the granular composition to fluorescence analysis. Formulation of chemical compounds into finished goods, in particular granulation, is usually required to achieve improved properties of the products, thus making them more commercially attractive. However, for biologically active compounds, granulation is often compulsory to the producers because the active compound must, until being applied in the intentional use, be separated from the surrounding environment to ensure the safe handling of the product. The amount of biologically active compound which can escape from the granulated product, e.g. in the form of dust, must be minimized to ensure that persons handling the product do not suffer any adverse effects from contact with the biologically active compound. Vice versa the active compound must be protected from the environment outside the granule to remain stabile and active once it is to be used. Once an active compound has been granulated it is known that one may further coat granules comprising biologically active compound with a coating agent which further suppress the release of active compound from the granule and further improve the stability of the active compound in the granule. Usually by increasing the thickness of the coating layer, it is possible to further improve granule properties.

One object of the invention is to provide methods, particularly in the form of an imaging system, for measuring quality parameters of a granular composition comprising a purified biologically active compound. Such a parameter may be for example the amount of active compound released from granules in the form of active dust, during or after the process for preparing the granular composition. Another parameter is for example the thickness and/or integrity of coating layers applied to the granules to suppress dust formation and increase the stability of the active compound which by the present invention could be measured during or after a process of coating of granules comprising a biologically active compound. Another object of the invention is to design a granulation apparatus and to select method setup, so that the method may be used on-line or in-line in the production of such granular compositions, and that the methods in real may time provide information about levels of dust comprising biologically active compound during processing of the granular composition.

We have found that for granular compositions comprising a purified biologically active compound both active compound confined in the surface regions of granules and active compound, which is present in the composition in the dust particles, e.g. as a result from release of active compound from the granules or as a result from insufficient granulation, can be evaluated by illuminating the composition with light capable of fluorescence excitation of a fluorescent marker, e.g. the biologically active compound itself, and detecting emitted light from the fluorescent marker. Accordingly, the present invention provides in a first aspect a method for fluorescence analysis comprising illuminating a granular composition comprising a purified biologically active is compound with light capable of fluorescence excitation of a fluorescent marker comprised in the granular composition, detecting light emitted from the fluorescent marker and predicting the amount of fluorescent marker in the granular composition accessible to fluorescence excitation. The amount of accessible fluorescent marker may be linked to a property of the granular composition, such as dust levels and/or coating thickness or coating integrity.

Further, in a second aspect, the invention provides a process for preparing granules comprising a purified biologically active compound, a fluorescent marker and optionally auxiliary granulation agents in a granulation apparatus said process comprising the step of performing fluorescence analysis in accordance with the first aspect of the invention (vide supra).

Still further, in a third aspect, the invention provides a granulation or coating apparatus comprising (a) a granulating or coating device comprising at least one chamber for granulating material or for coating of granulated material, (b) at least one detector capable of detecting emitted light, (c) means for projecting a source of illuminating light in form of a beam onto a portion of the material being processed, (d) means for canalising light emitted from illuminated material to the detector and (e) at least one device for selecting wavelength of the illuminating or emitted light.

Finally, in a fourth aspect the invention provides use of fluorescence analysis on granules comprising a purified biologically active compound.

### BRIEF DESCRIPTION OF THE DRAWINGS

FIG. 1: an example of an optical arrangement, wherein 1=detectors (CCD cameras), 2=light source, 3=band pass filters, 4=dichroic mirror beam splitters, 5=lenses, 6=funnel, 7=fluorescing granules passing the illuminating beam of light.

FIG. 2: an example of an on-line optical arrangement, wherein 1=picture showing the emitted light from the granules as recorded by a CCD camera, 2=CCD camera, 3=light source, 4=granules on a conveyer belt.

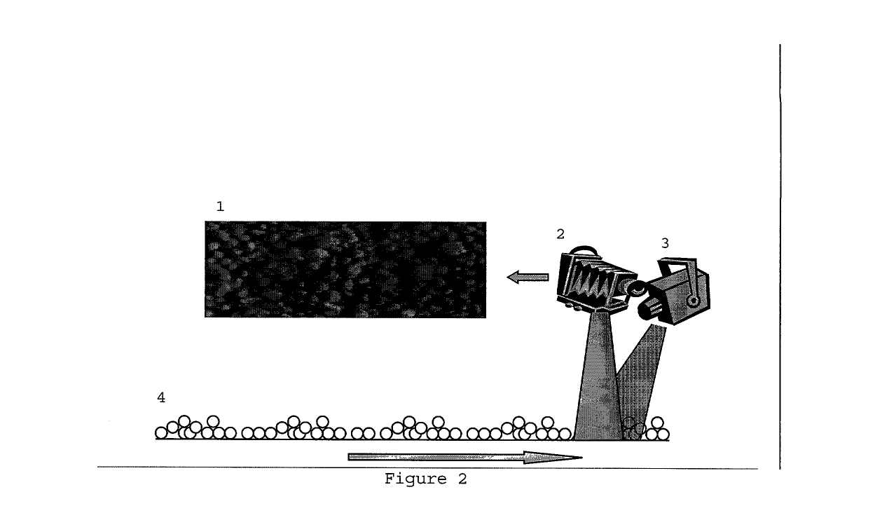

### DETAILED DESCRIPTION OF THE INVENTION

### Fluorescence Analysis

The present invention relates, as described, to a method for performing fluorescence analysis on a granular composition comprising an active compound.

The first step of the method comprises illuminating the granular composition with a beam of light, which can excitate a fluorescent marker comprised in the granular composition. In this process photons from the illuminating light will be absorbed by the fluorescent marker with the result, that electrons in the fluorescent compound gain energy and are brought into a specific increased energy level. The excitated fluorescent marker will, as the electrons return to their original ground state energy level, subsequently liberate at least some of the gained energy by emitting light photons of a wavelength characteristic for the energy difference between the increased energy level and the original energy level.

The second step of the method comprises detecting the emitted light from the granular composition with a detector, capable of converting the emitted light into an electronic signal.

The third step of the method comprises processing the electronic signal to correlate the amount of emitted lights to one ore more properties of the granular composition by predicting the amount of fluorescent marker in the granular composition accessible to fluorescence excitation.

The interpretation and meaning of all terms, pertaining to the basic principles of fluorescence analysis are known to the skilled person.

Illumination of the Granular Composition

The illumination of the granular composition may be carried out with any suitable light source delivering light capable of excitating the fluorescent marker in the granular composition. The light source may be e.g. a normal glow lamp, a more specialized xenon lamp or a stroboscope lamp.

The optical properties of the fluorescent marker compound may be known and to optimise the excitation of a known fluorescent marker it is preferred to select a light source delivering a substantial portion of light of wavelengths suitable for excitating the fluorescent marker. If it is desired (which it may be when using a known fluorescent marker) to avoid or limit excitation and emission from compounds other than the fluorescent marker, which may interfere with the analysis, it may be desired to filter the beam of light, so that only light of selected wavelengths illuminates the granular composition. This may be done with one or more beam splitters and one or more band pass filters, such as high and/or low band pass filters, or grate monochromators allowing only light with specific wavelengths to pass. These features are normally integrated in commercially available fluorescence analysers, e.g. from Perkin Elmer, USA. It is known to the skilled person that band pass filters and monochromators will allow passage of light having wavelengths within a narrow ranges, normally within a few nm, such as 0.5-10 nm. Thus the term monochromatic light is to be understood as light having wavelengths within the narrow range determined by the band pass filter or the grate monochromator.

In a particular embodiment the light illuminating the granular composition consist of 1-10 discrete monochromatic wavelengths, particularly 1-4 discrete monochromatic wavelengths. In particular the light illuminating the granular composition consist of one discrete monochromatic wavelength. In the step of illuminating the granular composition an optical arrangement may suitably be employed comprising e.g. mirrors, beam splitters (such as dichroic mirrors) and/or fiber optics to project the illuminating light onto the granular composition.

Detection of Emitted Light

The emitted light may be characteristic for the fluorescent marker or for chemical groups or constituents comprised in the fluorescent marker. As the fluorescent marker usually only emit light within one or more narrow ranges of wavelengths it is preferred to filter the emitted light, so that only emitted light within these ranges are allowed to reach the detector. This may be achieved by filtering the emitted light with one or more band pass filters or monochromators as described, supra. This may avoid or limit the amount of emitted light from compounds other than the fluorescent marker from reaching the detector and which may interfere with the analysis. Accordingly, in a particular embodiment only emitted light of 1-10 discrete monochromatic wavelengths are detected, particularly 1-4 discrete monochromatic wavelengths. In particular emitted light reaching the detector consists of one discrete monochromatic wavelength.

Further, as for the illumination, in the step of detecting the emitted light an optical arrangement may suitably be employed comprising e.g. mirrors, beam splitters (such as dichroic mirrors), fiber optics, and or means for focusing the emitted light (such as lenses) to project the emitted light into the detector.

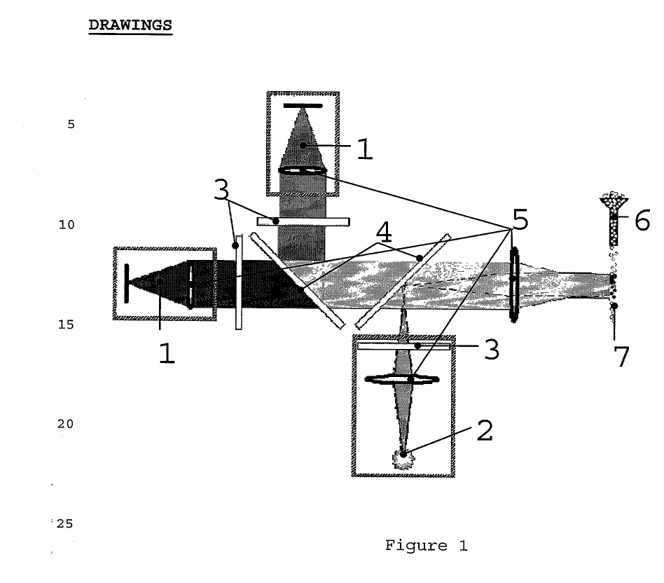

A variety of detectors may be applied to detect the emitted light. The detector may be a photo multiplication type detector, a photo diode or photo diode array, a line scan camera, a CCD camera, an ICCD camera or any other type suitably for detecting the emitted light. A particular detector may be a camera type detector, such as selected from the group of grey scale cameras, line scan cameras, photodiode arrays, CCD (charged Coupled Device) cameras and ICCD (Intensified CCD) cameras. In particular detectors may be CCD cameras and ICCD cameras, because they are more sensitive and enable formation of 2 dimensional images, showing the spatial distribution of the light emitting granules or dust particles. This is called fluorescence imaging in terms of the skilled analyst. In a particular embodiment two or more detectors may be used to record two or more selected wavelengths or two or more ranges of wavelengths simultaneously. This is desired if more than one fluorescent marker is to be measured or if a particular fluorescent marker emits light at different wavelengths. This is particularly achieved using an optical arrangement including one or more beam splitters and two and more band pass filters or monochromators. In a most particular embodiment an optical arrangement including two CCD cameras, dichroic mirrors and band pass filters and lenses as shown schematically in FIG. 1 is used.

Processing of Detected Signal

Conversion of the emitted light, in the detector, into an electronic signal and converting this signal into a measure, such as a number, from which a prediction of the amount of emitted light and the amount of fluorescent marker accessible to excitation may be inferred, is known to the skilled person, as analysers for making fluorescence analysis are abundantly available. The prediction may suitably be made by comparing the amount of the emitted light from an unknown granular composition with data on emitted light from a granular composition of known properties, and thus predicting in the unknown granular composition the amount of fluorescent marker accessible to excitation. The output of most detectors such as photo multiplication based types or some photo diode based types is an analogue signal. Most detectors such as many cameras, which comprise numerous single photo diode detectors, may have a build in analogue-digital converter capable of converting the analogue signal into a digital signal, which is more suitable for computerized data processing. Depending on the fluorescent marker and property of the granular composition one wishes to link to the amount of emitted light, the digital data arising from the emitted light may be subjected to processing. This processing is suitably performed in a computer system using software designed for such processes. Such software may be the LabView software as used in the examples herein or any other software providing the necessary capabilities for performing the desired data processing to link the amount of emitted light to a property of the granular composition. The data processing may include operations such as particle counting, gauging, pattern matching (grey scale and colour), statistics, thresholds, multivariate image analysis, AMT, blob analysis, area calculation, edge detection, morphology analysis, convolution, folding and unfolding, FFT, various filtering techniques e.g. median filtering—all techniques known to the skilled analyst, which are data processing functions included in commercially available software. In the process of transferring data to a computing unit the computing unit usually have to be equipped with hardware capable of acquiring the data from the detector for storage in the computing unit. Such hardware, e.g. data acquisition cards, is well known. When using a CCD or other type of camera producing 2 dimensional images of fluorescent granules it is also advantageous to use, in the computing unit, software capable of recording the 2 dimensional images in form of discrete digital still images. Such software is known as frame grabber programs. The speed at which such software is capable of recording images usually depends on the speed of the computing unit, and of for most fluorescence analysis purposes a speed of about 15 frames per second suffices. This means that 15 two-dimensional images are recorded per second.

### The Granular Composition

Physical Properties

The granular composition of the invention is a composition comprising the biologically active compound, a fluorescent marker, which may be the biologically active compound itself and optionally auxiliary granulation agents and coating agents processed into particles or granules. Accordingly, finished granules are the result of the processes and methods of the invention. The term “granules” are to be understood as a predominantly spherical or near spherical structure of a macromolecular size, particularly having an average size measure in the longest diameter between 20-2000 μm, more particularly between 100-1000 μm, most particularly between 200-800 μm. The spherical granules preferably have a ratio, (a):(b), between the diameter in the shortest dimension (a) and the diameter in the longest dimension (b) of the granule of between 1:1 to 1:5, particularly between 1:1 to 1:3.

The “size distribution” (PSD) of granules can be expressed in terms of the mass mean diameter of the individual particles. A mean mass diameter of D50 is the diameter at which 50% of the granules, by mass, have a smaller diameter, while 50% by mass have a larger diameter. The values D10 and D90 are the diameters at which 10% and 90%, respectively, of the granules, by mass, have a smaller diameter than the value in question. The “SPAN” indicates the breadth of the PSD and is expressed as: (D90−D10)/D50. For purposes of the present invention, the PSD of granules after granulation is normally as narrow as possible. Use of fluorescence analysis, in accordance with the present invention, for controlling the granulation process may aid in lowering of the PSD, and the SPAN of the granular composition after granulation is therefore particularly less than about 2.5, particularly less than about 2.0, more particularly less than about 1.5, and most particularly less than about 1.0.

The granules are particularly coated with a coating agent forming a, particularly homogenous, coherent and continuous, layer around the granules. The term coating agent as used herein is to be understood as single coating compound or a mixture of coating compounds. Coated granules thus consist of a granule core and a granule coating. Particularly the coating layer is relatively thick in order to further reduce dusting and improve stability of the biologically active compound. The coating thickness may be described by the ratio between the average diameter of a coated granule core and the average diameter of an uncoated granule core (hereinafter abbreviated D_{G}/D_{C}), i.e. the average diameter of the coated granule divided by the average diameter of the granule core only. If for example a granule core having a diameter of 100 μm is coated with a coating layer 200 μm thick, the granule would have a diameter of (200+100+200)=500 μm and D_{G}/D_{C} is 500 μm/100 μm=5. Coated granules of the invention particularly have a D_{G}/D_{C} of at least 1.1, which means that the thickness of the coating is at least 5% of the average granule core diameter. A more particular D_{G}/D_{C} is at least 1.5, more particularly at least 2, more particularly at least 2.5, more particularly at least 3, most particularly at least 4. D_{G}/D_{C} is however particularly below about 100, particularly below about 50, more particularly below 25, and most particularly below 10. A most particular range for D_{G}/D_{C} is about 4 to about 6.

Furthermore, in the present invention the coating is substantially enzyme-free, The term “substantially enzyme free ” as used herein about a coating means that there is less than 5 mg of enzyme per gram coating agent.

Considering the materials making up granules of the invention (vide infra) these are, as opposed to transparent materials such as glass, in general impassable to visual light, e.g. a person will usually not be able to see-through the granules. However, when subjecting granules comprising a biologically active compound of the art to excitation light we have discovered that such excitation light may penetrate the surface of the granule and excitate a suitable fluorescent compound present at and/or near the surface of the granule and that emitted light from such fluorescent compound may escape the granule so that it can be received by a detector.

Further, the content of moisture in the granules is typically be less than 20 (w/w)%, particularly less than 15 (w/w)%, more particularly less than 10 (w/w)%, such as in the range of 4 to 8 (w/w)%.

Construction

The granules may be constructed by any granulation method known in the art.

The construction of the granules of the invention may be divided into the following non-exhaustive categories:

a) Spray dried granules, wherein a liquid solution containing the biologically active compound is atomised in a spray dryer to form small droplets which during their way down the dryer dry to form a granular material comprising the biologically active compound. Very small granules can be produced this way (Michael S. Showell (editor); Powdered detergents; Surfactant Science Series; 1998; vol. 71; page 140-142; Marcel Dekker). For these granules the biologically active compound is intimately mixed with any other auxiliary granulation agents present in the liquid solution.

b) Layered granules, wherein the biologically active compound is coated as a layer around a pre-formed core particle, wherein an solution containing the biologically active compound, and particularly auxiliary granulation agents, is atomised, typically in a fluid bed apparatus wherein the pre-formed core particles are fluidised, and the solution of biologically active compound adheres to the core particles and dries up to leave a layer of dry biologically active compound on the surface of the core particle. Granules of a desired size can be obtained this way if a useful core particle of the desired size can be found. This type of granules is described in e.g. WO 97/23606

c) Absorbed core granules, wherein rather than coating the biologically active compound as a layer around the core, the biologically active compound is absorbed onto and/or into the surface of the core. Such a process is described in WO 97/39116.

d) Extruded or pelletized granules, wherein a paste containing the biologically active compound is pressed into granules in a mould or under pressure is extruded through a small opening and cut into granules which are subsequently dried. Such granules usually have a considerable size because of the material in which the extrusion opening is made (usually a plate with bore holes) sets a limit on the allowable pressure drop over the extrusion opening. Also, very high extrusion pressures when using a small opening increase heat generation in the paste, which may be harmful to the biologically active compound. (Michael S. Showell (editor); Powdered detergents; Surfactant Science Series; 1998; vol. 71; page 140-142; Marcel Dekker)

e) Spray cooled granules, wherein a powder of biologically active compound is suspended in molten wax and the suspension is sprayed, e.g. through a rotating disk atomiser, into a cooling chamber where the droplets quickly solidify (Michael S. Showell (editor); Powdered detergents; Surfactant Science Series; 1998; vol. 71; page 140-142; Marcel Dekker). For these granules the biologically active compound is intimately mixed with the wax instead of being concentrated on its surface. Also U.S. Pat. No. 4,016,040 and U.S. Pat. No. 4,713,245 are documents relating to this technique

f) High shear mixer granules, wherein a liquid containing the biologically active compound is added to a dry powder composition of auxiliary granulation agent. The liquid and the powder in a suitable proportion are mixed and as the moisture of the liquid is absorbed in the dry powder, the components of the dry powder will start to adhere and agglomerate and granules will build up, forming granules comprising the biologically active compound. For these granules the active compound is intimately mixed with the auxiliary granulation agents. Such a process is described in U.S. Pat. No. 4,106,991 (NOVO NORDISK) and related documents EP 170360 B1 (NOVO NORDISK), EP 304332 B1 (NOVO NORDISK), EP 304331 (NOVO NORDISK), WO 90/09440 (NOVO NORDISK) and WO 90/09428 (NOVO NORDISK).

Dust Particles in Granular Compositions

Dust particles, which may be present in a granular composition, may be characterised in that they are fragments of whole granules, which usually have a considerably smaller size than the granules and do not possess the characteristic spherical shape of the granules. Dust particles typically have an irregular non-spherical and abrupt structure such as rod or flake shaped. Dust particles are typically much smaller than the average size of granules, and most dust particles are, depending on the granular composition less than 20 μm in diameter.

Dust particles being fragments of whole granules possess the same in-transparency and usually have about the same moisture content as intact granules, supra.

### Compounds in the Granular Composition

Biologically Active Compounds

The granular composition of the invention comprises a biologically active compound, in particular a purified biologically active compound. The term biologically active compound as used herein is to be understood as any compound, which is active in a biological system such as compounds, which interfere with and/or modifies biological pathways or biological reactions. The term “purified” as used herein is to be understood as biologically active compounds, which before granulation has been subjected to one or more purification step to remove e.g. excess material and/or to concentrate the active compound. In the case the active compound is prepared by a microbiological fermentation process purification particularly includes step selected from filtering, ultra-filtration, flocculation, sedimentation, evaporation, extraction and the like, to remove biomass and other undesired matter including water to yield a mixture which is enriched in the biologically active compound.

Biologically active compounds include among others organic compounds such as bio-catalysts, therapeutic agents, herbicides, pesticides and fungicides. In particular the biologically active compound is producible by fermenting a microorganism producing the active compound.

In particular compounds are those among proteins and peptides, more particularly catalytic proteins, i.e. enzymes, because proteins such as enzymes are used in vast volumes in the industry and are known to cause adverse allergy reactions in humans or animal when exposed to such proteins. Furthermore, enzymes are widely used in household products such as detergents for removing soil of a biological origin, and many industrial processes involves human handling of the enzymes. The enzyme may be any enzyme for which it is desired to separate the enzyme from the surrounding environment through granulation of the enzymes.

The enzyme classification employed in the present specification with claims is in accordance with Recommendations (1992) of the Nomenclature Committee of the International Union of Biochemistry and Molecular Biology, Academic Press, Inc., 1992.

Accordingly the types of enzymes which may appropriately be incorporated in granules of the invention include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Particular oxidoreductases in the context of the invention are peroxidases (EC 1.11.1), laccases (EC 1.10.3.2) and glucose oxidases (EC 1.1.3.4)], while particular transferases are transferases in any of the following sub-classes:

a) Transferases transferring one-carbon groups (EC 2.1);

b) transferases transferring aldehyde or ketone residues (EC 2.2); acyltransferases (EC 2.3);

c) glycosyltransferases (EC 2.4);

d) transferases transferring alkyl or aryl groups, other that methyl groups (EC 2.5); and

e) transferases transferring nitrogenous groups (EC 2.6).

A most particular type of transferase in the context of the invention is a transglutaminase (protein-glutamine γ-glutamyltransferase; EC 2.3.2.13).

Further examples of suitable transglutaminases are described in WO 96/06931 (Novo Nordisk A/S).

Particular hydrolases in the context of the invention are: Carboxylic ester hydrolases (EC 3.1.1.-) such as lipases (EC 3.1.1.3); phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as α-amylases (EC 3.2.1.1); peptidases (EC 3.4, also known as proteases); and other carbonyl hydrolases].

In the present context, the term “carbohydrase” is used to denote not only enzymes capable of breaking down carbohydrate chains (e.g. starches) of especially five- and six-membered ring structures (i.e. glycosidases, EC 3.2), but also enzymes capable of isomerizing carbohydrates, e.g. six-membered ring structures such as D-glucose to five-membered ring structures such as D-fructose.

Carbohydrases of relevance include the following (EC numbers in parentheses):

alpha-amylases (3.2.1.1), beta-amylases (3.2.1.2), glucan 1,4-alpha-glucosidases (3.2.1.3), cellulases (3.2.1.4), endo-1,3(4)-beta-glucanases (3.2.1.6), endo-1,4-beta-xylanases (3.2.1.8), dextranases (3.2.1.11), chitinases (3.2.1.14), polygalacturonases (3.2.1.15), lysozymes (3.2.1.17), beta-glucosidases (3.2.1.21), alpha-galactosidases (3.2.1.22), beta-galactosidases (3.2.1.23), amylo-1,6-glucosidases (3.2.1.33), xylan 1,4-beta-xylosidases (3.2.1.37), glucan endo-1,3-beta-D-glucosidases (3.2.1.39), alpha-dextrin endo-1,6- alpha-glucosidases (3.2.1.41), sucrose alpha-glucosidases (3.2.1.48), glucan endo-1,3-alpha-glucosidases (3.2.1.59), glucan 1,4-beta-glucosidases (3.2.1.74), glucan endo-1,6-beta-glucosidases (3.2.1.75), arabinan endo-1,5-alpha-L-arabinosidases (3.2.1.99), lactases (3.2.1.108), chitosanases (3.2.1.132) and xylose isomerases (5.3.1.5).

Examples of commercially available oxidoreductases (EC 1.-.-.-) include GLUZYME™ (enzyme available from Novo Nordisk A/S).

Examples of commercially available proteases (peptidases) include KANNASE™, EVERLASE™, ESPERASE™, ALCALASE™, NEUTRASE™, DURAZYM™, SAVINASE™, PYRASE™, PANCREATIC TRYPSIN NOVO (PTN), BIO-FEED™ PRO and CLEAR-LENS™ PRO (all available from Novo Nordisk A/S, Bagsvaerd, Denmark).

Other commercially available proteases include MAXATASE™, MAXACAL™, MAXAPEM™, OPTICLEAN™ and PURAFECT™ (available from Genencor International Inc. or Gist-Brocades).

Examples of commercially available lipases include LIPOPRIME™ LIPOLASE™, LIPOLASE™ ULTRA, LIPOZYME™, PALATASE™, NOVOZYM™ 435 and LECITASE™ (all available from Novo Nordisk A/S).

Other commercially available lipases include LUMAFAST™ (Pseudomonas mendocina lipase from Genencor International Inc.); LIPOMAX™ (Ps. pseudoalcaligenes lipase from Gist-Brocades/Genencor Int. Inc.; and Bacillus sp. lipase from Solvay enzymes.

Examples of commercially available carbohydrases include ALPHA-GAL™, BIO-FEED™ ALPHA, BIO-FEED™ BETA, BIO-FEED™ PLUS, BIO-FEED™ PLUS, NOVOZYME™ 188, CELLUCLAST™, CELLUSOFT™, CEREMYL™, CITROZYM™, DENIMAX™, DEZYME™, DEXTROZYME™, FINIZYM™, FUNGAMYL™, GAMANASE™, GLUCANEX™, LACTOZYM™, MALTOGENASE™, PENTOPAN™, PECTINEX™, PROMOZYME™, PULPZYME™, NOVAMYL™, TERMAMYL™, AMG™ (Amyloglucosidase Novo), MALTOGENASE™, SWEETZYME™ and AQUAZYM™ (all available from Novo Nordisk A/S). Further carbohydrases are available from other suppliers.

The amount of enzyme to be incorporated in a granule of the invention will depend on the intended use of the granule. For many applications, the enzyme content will be as high as possible or practicable.

The content of enzyme (calculated as pure enzyme protein) in a granule of the invention will typically be in the range of from about 0.5% to 50% by weight of the enzyme-containing granule.

Auxiliary Granulation Agents

The granules of the invention particularly contains auxiliary granulation agents for purposes such as aiding the formation of granules, controlling density and volume of granules, controlling amount of active compound in the granules, stabilising the active compound and the like.

Auxiliary granulating agents may include but is not limited to:

a) Fillers such as fillers conventionally used in the field of granulation e.g. water soluble and/or insoluble inorganic salts such as finely ground alkali sulphate, alkali carbonate and/or alkali chloride), clays such as kaolin (e.g. Speswhite™, English China Clay), bentonites, talcs, zeolites, and/or silicates.

b) Binders such as binders conventionally used in the field of granulation e.g. binders with a high melting point or no melting point at all and of a non waxy nature e.g. polyvinyl pyrrolidon, dextrins, polyvinylalkohol, cellulose derivatives, for example hydroxypropyl cellulose, methyl cellulose or CMC. A suitable binder is a carbohydrate binder such as Glucidex 21D available from Roquette Freres, France.

c) Fiber materials such as fibers conventionally used in the field of granulation. Pure or impure cellulose in fibrous form can be sawdust, pure fibrous cellulose, cotton, or other forms of pure or impure fibrous cellulose. Also, filter aids based on fibrous cellulose can be used. Several brands of cellulose in fibrous form are on the market, e.g. CEPO and ARBOCELL. In a publication from Svenska Trämjolsfabrikerna AB, “Cepo Cellulose Powder” it is stated that for Cepo S/20 cellulose the approximate maximum fiber length is 500 μm, the approximate average fiber length is 160 μm, the approximate maximum fiber width is 50 μm and the approximate average fiber width is 30 μm. Also, it is stated that CEPO SS/200 cellulose has an approximate maximum fiber length of 150 μm, an approximate average fiber length of 50 μm, an approximate maximum fiber width of 45 μm and an approximate average fiber width of 25 μm. Cellulose fibers with these dimensions are very well suited for the purpose of the invention. The words “Cepo” and “Arbocel” are Trademarks. Particular fibrous cellulose is Arbocel™ BFC200. Also synthetic fibres may be used as described in EP 304331 B1 and typical fibres may be made of polyethylene, polypropylene, polyester, especially nylon, polyvinylformat, poly(meth)acrylic compounds.

d) Liquid agents such as conventionally used in the field of granulation. A liquid agent is used in conventional mixer granulation processes for enabling the build up or agglomeration of the conventional granulating component particles into granules. The liquid agent is water and/or a waxy substance. The liquid agent is always used in a liquid phase in the granulation process but may later on solidify; the waxy substance if present, therefore, is either dissolved or dispersed in the water or melted. By the term “waxy substance” as used herein is meant a substance which possesses all of the following characteristics 1) the melting point is between 30 and 100° C., particularly between 40 and 60° C., 2) the substance is of a tough and not brittle nature, and 3) the substance possesses a certain plasticity at room temperature. Both water and waxy substance are liquid agents, i.e. they are both active during the formation of the granules; the waxy substance stays as a constituent in the finished granules, whereas the majority of the water is removed during a drying step. Examples of waxy substances are polyglycols, fatty alcohols, ethoxylated fatty alcohols, mono-, di- and triglycerolesters of higher fatty, acids, e.g. glycerol monostearate, alkylarylethoxylates, and coconut monoethanolamide.

If a high amount of waxy substance is used, relatively little water should be added, and vice versa. Thus, the liquid agent can be either water alone, waxy substance alone or a mixture of water and waxy substance. When a mixture of water and waxy substance is used the water and the waxy substance can be added in any sequence, e.g. first the water and then the waxy substance, or first the waxy substance and then the water or a solution or suspension of the waxy substance in the water. Also, when a mixture of water and waxy substance is used, the waxy substance can be soluble or insoluble (but dispersible) in water. If water is used a liquid agent it may not be a part of the finished mixer granule as usually most of the water is dried off at a subsequent drying of the mixer granules.

e) Enzyme stabilizing or protective agents such as conventionally used in the field of granulation. Stabilizing or protective agents may fall into several categories: alkaline or neutral materials, reducing agents, antioxidants and/or salts of first transition series metal ions. Each of these may be used in conjunction with other protective agents of the same or different categories. Examples of alkaline protective agents are alkali metal silicates, -carbonates or bicarbonates, which provide a chemical scavenging effect by actively neutralizing e.g. oxidants. Examples of reducing protective agents are salts of sulfite, thiosulfite or thiosulfate, while examples of antioxidants are methionine, butylated hydroxytoluene (BHT) or butylated hydroxyanisol (BHA). Most particular agents are salts of thiosulfates, e.g. sodium thiosulfate. Also enzyme stabilizers may be borates, borax, formates, di- and tricarboxylic acids and reversible enzyme inhibitors such as organic compounds with sulfhydryl groups or alkylated or arylated boric acids.

f) Cross linking agents such as conventionally used in the field of granulation. Cross linking agents may be enzyme-compatible surfactants e.g. ethoxylated alcohols, especially ones with 10 to 80 ethoxy groups.

Further, suspension agents, mediators (for boosting bleach action upon dissolution of the granule in e.g. a washing application or mediators for enzymes) and/or solvents may be incorporated as auxiliary granulating agents.

Coating Agents

The coating comprises one or more conventional coating agents components such as described in WO 89/08694, WO 89/08695, EP 270 608 B1 and/or WO 00/01793. Other examples of coating agents may be found in U.S. Pat. No. 4,106,991, EP 170360, EP 304332, EP 304331, EP 458849, EP 458845, WO 97/39116, WO 92/12645A, WO 89/08695, WO 89/08694, WO 87/07292, WO 91/06638, WO 92/13030, WO 93/07260, WO 93/07263, WO 96/38527, WO 96/16151, WO 97/23606, U.S. Pat. No. 5,324,649, U.S. Pat. No. 4,689,297, EP 206417, EP 193829, DE 4344215, DE 4322229 A, DD 263790, JP 61162185 A and/or JP 58179492. Especially the salt coatings described in WO 00/01793 are useful for coatings in the present invention.

The coating agent may be selected from the list of auxiliary granulation agents described, supra. Further coating agents may be selected the following non-limiting list of polymers, chlorine scavengers, plasticizers, pigments, lubricants (such as surfactants or antistatic agents) and fragrances.

Polymers useful in coating layers include vinyl polymers or vinyl co-polymers such as polyvinyl alcohol (PVA) and/or polyvinyl pyrrolidone or derivatives thereof. Also included are isoptalic acid polymers.

Plasticizers useful in coating layers in the context of the present invention include, for example: polyols such as sugars, sugar alcohols, or polyethylene glycols (PEGs) having a molecular weight less than 1000; urea, phthalate esters such as dibutyl or dimethyl phthalate; and water.

Suitable pigments include, but are not limited to, finely divided whiteners, such as titanium dioxide or kaolin, coloured pigments, water soluble colorants, as well as combinations of one or more pigments and water soluble colorants.

As used in the present context, the term “lubricant” refers to any agent, which reduces surface friction, lubricates the surface of the granule, decreases tendency to build-up of static electricity, and/or reduces friability of the granules. Lubricants can also play a related role in improving the coating process, by reducing the tackiness of binders in the coating. Thus, lubricants can serve as anti-agglomeration agents and wetting agents. Examples of suitable lubricants are polyethylene glycols (PEGs) and ethoxylated fatty alcohols.

In embodiments aimed primarily at granules for detergent formulations, different “functional” components could be added to the coating such as TAED, CMC, bleach, OBA, surfactants, perfume as well as other functional components used in detergent formulations known to the person skilled in the art. The coating may also optionally comprise functional components selected for their specific use in the, pharmaceutical industry, agriculture, foodstuffs industry, baking industry, additives industry, feed industry, detergents industry or other industries where granules comprising a biologically active compound can be used.

In a particular embodiment of the invention the granule of the invention is coated with a protective coating having a high constant humidity of at least 81% such as described in WO 89/08694, which is hereby incorporated by reference. Accordingly, the coating should, in certain embodiments, act as a moisture and/or bleach barrier to stabilise the biologically active compound in the core. Furthermore, in alternative embodiments, the coating unit acts as a mechanical barrier during mechanical processes such as dosing or tabletting. In certain embodiments, the coating unit is sufficiently compressible and flexible for the core to withstand a tabletting process, both in a structural sense and with regards to biological activity of the active compound. This is potentially most applicable for detergent formulations.

In a particular embodiment the coating agent absorbs light from the excitation source and/or emitted from a fluorescent marker in the granule, so that when an area of the granule surface is coated with the coating agent a reduction in detected light emitted from this area is achieved.

The Fluorescent Marker

The fluorescent marker comprised the granular composition of the invention may be any compound exhibiting fluorescence when being illuminated. The fluorescent marker is particularly organic and exhibiting fluorescence when illuminated with light in the X-ray, ultra violate and/or visible regions of the electromagnetic spectrum, e.g. light having wavelengths between 10-700 nm, more particular light in the ultra violate region, i.e. 10-380 nm.

Furthermore, the fluorescent marker comprised the granular composition of the invention is capable upon excitation to emit light in the ultra violate, visible and/or near infrared regions of the electromagnetic spectrum, i.e. suitably between 185-2600 nm.

The fluorescent marker may belong to the group of biologically active compounds, auxiliary granulation agents and coating agents or it may be a compound added to the granular composition with the sole purpose of performing the fluorescence analysis of the invention. It is however from a cost saving point of view preferred that the fluorescent marker is the biologically active compound itself or it is an auxiliary granulation agent.

Depending on the property of the granulated composition to be evaluated by the fluorescence analysis, e.g. dust properties or coating thickness, different fluorescent marker may be chosen. For evaluation of dust properties it is for example more suitable to chose the biologically active compound as the fluorescent marker because it is necessary to assess the amount of potentially damaging active compound in dust particles present in the granular composition, while for evaluation of coating thickness it may be possible to choose a suitable auxiliary granulation agent as fluorescent marker.

In the case the biologically active compound is the fluorescent marker, especially those belonging to the particular group of proteins and/or peptides, in which it is aromatic amino acids residues such as tyrosine and tryptophan that are responsible for the fluorescence emission, it is preferred, in the fluorescence analysis, to illuminate the granular composition with a light source delivering ultra violet light (UV-light), particularly delivering a substantial portion of UV-light having wavelengths between 10-380 nm, more particularly between 200-400 nm or 200-300 nm, most particularly between 260-280 nm. In this case it is further preferred to detect only emitted light in the range of 200-700 nm, such as from 400-700 nm, or from 300-400 nm, especially light from 280-360 nm or from 325-375 nm. Also, in this case it is required to choose a detector, such as a CCD camera, capable of detecting emitted light having these wavelengths.

In case the fluorescent marker is one of the auxiliary granulation agents, it is preferred, in the fluorescence analysis, to illuminate to granular composition with a light source delivering light having wavelengths between 350-550 nm, more particularly between 375-425 nm. Also in this case it is required to choose a detector, such as a CCD camera, capable of detecting emitted light having these wavelengths. In the process of making granules one may also add a fluorescent marker to the process, which only serves the purpose of being a fluorescent marker for the fluorescence analysis. A wide range of suitable fluorescent compound are available e.g. from Molecular Probes, USA.

### Fluorescence Analysis in Granulation and Coating Processes

The present invention also encompass processes for preparing granular compositions comprising a biologically active compound and optionally auxiliary granulation agents in a granulation apparatus using the above mentioned method of fluorescence analysis to predict properties of the granular composition and control and improve the preparation process.

Accordingly, the present invention provides a process for preparing granules comprising a biologically active compound and optionally auxiliary granulation agents in a granulation apparatus said process comprising the step of performing fluorescence analysis on a fluorescent marker comprised in the granular composition as described, supra, on the granules forming in the granulator.

In a particular embodiment the fluorescence analysis is carried out during the formation of granules in the granulation process, particularly on-line, meaning that the fluorescence analysis is performed more than one time in real time during the granulation process with a suitable rate of repetition. The repetition rate will, inter alia, depend on the data processing of data from the detector(s). In the particular embodiment of using CCD or ICCD cameras about 15 measurements of the emitted light per second is recorded in form of two-dimensional images in the granulation process. The term “formation of granules” includes also coating granules with a coating layer. In this embodiment the process also particularly comprises the step of changing at least one process parameter as a result of the fluorescence analysis. The process parameter to be changed may be any parameter influencing the granulation process and/or the properties of the formed granules. These parameters may be supply of granulation material, i.e. biologically active compound and/or auxiliary granulation agents and/or coating agent to the granulator, supply of gas to the granulator, temperature in the granulator, pressure in the granulator, pH in the granulator and mechanical force conferred to the granulation material. The process parameter may be changed manually or though an automated system, cf. granulation apparatus.

In a further embodiment fluorescence analysis in accordance with the invention may also suitably be used to control dusting properties of finished granular compositions after granulation. Accordingly, the invention further provides a method for fluorescence analysis of active dust in a granular composition comprising a biologically active compound. Using this method, granular compositions, which do not meet the desired quality with respect to dust, may be discarded or reprocessed.

In a further additional embodiment fluorescence analysis in accordance with the invention may also suitably be used to control coating thickness and/or homogeneity of finished granular compositions after granulation. Accordingly, the invention further provides a method for fluorescence analysis of coating thickness in a composition of coated granules comprising a biologically active compound. Using this method granular compositions, which do not meet the desired quality with respect to coating thickness may be discarded or reprocessed.

The imaging system can, as said be used for evaluation of a quality parameter, such as active dust values or coating thickness. In one embodiment the evaluation is performed on-line during for example a coating process. The evaluation is performed by recording, in a particular by means of a camera, images of emitted light from granules subjected to an excitation light source and comparing the recorded images with images recorded of samples (reference samples) with known values of a quality parameter. The fluorescent images of the reference samples and the corresponding quality parameters are used to provide a calibration model, such as a Partial Least Square model or any other modelling system suitable for making calibration models of image data. The model may then used to predict estimates of a quality parameter from the fluorescent images of an unknown sample.

Thus the present invention also includes a method for estimating a quality parameter of a granular composition comprising a purified biologically active comprising the steps of:

a) providing a calibration model by illuminating a granular composition comprising a purified biologically active compound having a known quality parameter with light capable of fluorescence excitation of a fluorescent marker comprised in the granular composition, recording one or more images of the light emitted from the granular composition of a known quality and subjecting recorded images to data processing, particularly in the form of partial least squares data processing, to form a calibration model,

b) illuminating a unknown granular composition comprising a purified biologically active compound with light capable of fluorescence excitation of a fluorescent marker comprised in the granular composition, recording at least one image of the light emitted from the unknown granular composition,

c) comparing at least one image of the unknown granular composition with the calibration model and

d) estimating the quality parameter of the unknown granular composition.

### Granulation Apparatus

Also included in the scope of the invention is a granulation and/or coating apparatus comprising means for performing fluorescence analysis on granular compositions in accordance with the invention. Accordingly, the invention provides a granulation or coating apparatus comprising:

(a) a granulation or coating device comprising at least one chamber for processing material into granules or coated granules,

(b) an optical arrangement for performing fluorescence analysis comprising a light source for illumination of material being processed, at least one detector capable of detecting light emitted from the material being processed, means for projecting illuminating light onto a portion of the material being processed, means for projecting light emitted from illuminated material to the detector and at least one device for filtering light.

The granulation or coating device may be any conventional granulation device, in particular it may be selected from fluid bed granulators or coaters, high shear mixer granulators, spray dryers, a spray coolers and extruders.

In the optical arrangement the light source is particularly a normal glow lamp, a more specialized xenon lamp or a stroboscope lamp, particularly capable of delivering light having wavelengths between 10-700 nm, more particularly light in the ultra violate region, i.e. 10-380 nm.

In the optical arrangement the detector is particularly a camera type detector, more particularly a line-scan camera, a CCD or an ICCD camera.

Optionally the optical arrangement comprises means for focusing emitted light, such as lenses.

The means for projecting illuminating light onto the material being processed and projecting emitted light from said material to the detector includes one or more of fiber optics, mirrors, lenses, beam splitters and the like.

The optical arrangement includes at least one filtering device for filtering the illuminating and/or emitted light. In a particular embodiment this device is a band pass filter or a grate monochromator. In one embodiment one or more filtering devices are positioned so than only the emitted light is filtered and so that the emitted light must pass the filter(s) before reaching the detector(s). In another embodiment at least two filtering devices are positioned so that both the illuminating light and the emitted light is filtered. Most preferred filters are those selecting wavelength as described for the particular fluorescent markers, supra. In the case two detectors are used the optical arrangement also includes at least one beam splitter such as dichroic mirrors.

In a most particular embodiment the optical arrangement includes a stroboscope light source, 2 CCD camera detectors, one band pass filter for filtering illumination light, two band pass filters for filtering emitted light, lenses and two dichroic mirror beam splitters as depicted in FIG. 1.

In one particular embodiment the projecting means include means for projecting illuminating light through an opening in the chamber onto a portion of material being processed in the chamber and projecting emitted light from this material in the chamber to the detector.

In a more particular embodiment the granulation apparatus further comprise means for providing a purge stream of material from the chamber. In this case the optical arrangement is positioned to allow fluorescence analysis of material in the purge stream rather than on material present in the chamber. One reason for preferring this embodiment is that granulation processes usually involves considerable wear and tear of the granulation equipment. When granulating a biologically active compound such as an enzyme, some auxiliary granulation agents may be clays or other inorganic substances. These substances may have a significant sanding effect on the granulation equipment. Accordingly, when granulating in e.g. a mixer it is not unusual to observe that several millimetres of steel is sanded of the surfaces on the interior parts of the granulation equipment per year. This magnitude of wear and tear may be very detrimental to the sensitive equipment of the optical arrangement. The granules in the purge stream may suitably be recycled and this way the fluorescence analysis does not interfere with the granulation process. The purge stream may suitably lead the granules past a part of the purge stream transparent to light wherein the projection of light may occur. As an example the purge stream may be transported from the granulation chamber, through a transporting system, which e.g. may include one or more elements selected from chutes, pumps, pipe, conveyor belts, cyclones and the like, to the optical arrangement. The fluorescence analysis may suitably occur at a point in the transporting system, where the granulated product can be accessed by the illuminating light, and from which point the emitted light may reach the detector(s) . For example such point may be a point where part of e.g. a pipe material is replaced with a transparent material such as glass, quartz or a polymeric material. Particularly, at the point where the fluorescence analysis occurs the means for providing the purge stream includes means for forming a single layer of granules, so that no or little overlapping occurs in the detection from granules, which superimpose each other at the moment of detection. This may be achieved by loading the granulated product onto a tilted (non-horizontal) vibrating surface (e.g. a vibrating chute), where the area of the surface, the speed at which the granules are loaded, and the transport speed of the granules over the vibrating surface (the tilting angle) are adjusted so that the area of the shaking surface is always larger than the area of the granules present on the surface. This way the granules will form a substantial single (mono) layer of granules transported over surface. The fluorescence analysis may be performed at any place of this single layer of granules. The granules may particularly also leave the vibrating surface as a single layer as they fall over one or more edges of the vibrating surface (comparable to a waterfall) and in order to avoid reflections from the vibrating surface the fluorescence analysis particularly take place at some point after the granules leaves the vibrating surface, but while they still maintain a single layer of granules. By measuring on granules forming a single layer of granules overlapping is avoided and the emitted light from the granules may be more precisely focused as the granules is primarily distributed in only two dimensions. This way, sharply focused images of nearly all individual granules passing the point of fluorescence analysis may be obtained using a two-dimensional detector such as a CCD camera.

As indicated above the optical arrangement is suitably connected to the granulation or coating device to enable on-line or at-line fluorescence analysis of granular compositions. On-line analysis is to be understood as analysis performed on granules as they are actually being granulated, e.g. by analyzing granules in the granulator or in a recycled purge stream. At-line analysis is to be understood as analysis performed down stream after the granulation process (e.g. at the outlet) or on non-recycled samples taken from the granulator during granulation.

The granulation apparatus may comprise other elements such computing units for processing data from detectors, optionally equipped with specialised data handling hardware and software. The granulation apparatus may also comprise control units linked to the computing units for controlling and adjusting the granulation process based on the results of the fluorescence analysis. A control unit may be a PLC or other equipment capable of receiving data from a computing unit and converting these data into output controlling one or more hardware devices influencing the granulation process, such as feed streams, speed, temperature, airflows etc.

The procedure for carrying out the present is demonstrated in the following experiments. The experiments are only examples of one embodiment of the invention and should in no way be interpreted as limiting to the scope of the invention.

### EXAMPLES

### Example 1

Fluorescence Analysis on Raw Materials for Making Enzyme Granules:

Fluorescence analysis of eight different raw materials for enzyme granulation and enzyme concentrate where measured on a LS50B (Perkin Elmer) instrument. The raw materials were excitated with light of the internal source of different wavelengths from 230-500 nm in 10 nm steps and emission from the materials was recorded from 270-700 nm in steps of 1 nm. Slit gap in the Perkin Elmer instrument for both excitation and emission was 4 nm. The fluorescence was measured by illuminating the raw material placed in a quartz container (cuvette) and measuring fluorescence emission from the raw materials at an angle displaced 22.5 degrees from the direction of the illuminating light as required by the instrument design.

Several raw materials fluoresced significantly but the enzyme concentrate had a uniquely a very intensive emission at about 350 nm.

### Example 2

Fluorescence Analysis on Enzyme Granules

Fluorescence analysis of uncoated enzyme granules as well as coated granules were measured on a LS50B (Perkin Elmer) instrument. The granules were excitated with light of the internal source of different wavelengths from 230-500 nm in 10 nm steps and emission from the materials was recorded from 270-700 nm in steps of 1 nm. Slit gap in the Perkin Elmer instrument for both excitation and emission was 4 nm. The fluorescence was measured by illuminating the raw material placed in a quartz container (cuvette) and measuring fluorescence emission from the raw materials at an angle displaced 22.5 degrees from the direction of the illuminating light as required by the instrument design.

The results showed a peak of emission around 350 nm corresponding to the enzyme concentrate. Another strong peak occurred between 450-500 nm due to fluorescent compounds among granulation auxiliaries. The results show the feasibility of detecting enzymes in the granules.

### Example 3

On-line Fluorescence Analysis of Granules:

Fluorescence analysis of uncoated enzyme granules as well as coated granules was measured on using a optical arrangement as shown in FIG. 1. Two camera detectors of the type Donpisha “Progressive Camera Module” XC-8500CE 1/2″ ITCCD 782(H) x 582(V), with a CCIR Additional 35 mm lens at sample introduction point was used. The light source was an Oriel Xenon Flash Lamp 60000 w/ Oriel attachment 60008 and Oriel Power Supply 68826. Illuminating light was filtered using a band pass filter to produce a beam of light having a wavelength of 450 nm. Before reaching the cameras the emitted light was split into two beams by a dichroic mirror beam splitter and each beam was filtered by a band pass filters. One filter allowed passage of 530 nm light (green filter) and the other filter allowed passage of 620 nm light (red filter).

The illumination and detection of emitted light was carried out by loading enzyme granules into a funnel having an outlet of proportion allowing passage of the granules and performing the analysis on granules leaving the funnel immediately after the funnel outlet.

The signal from the detectors was transferred to a computing unit (ordinary personal computer) equipped with data acquisition hardware, DAQ and SCB-68 breadboard and image processing software, Labview 4.1.1 IMAQ vision and All-purpose Labview all from National Instruments to produce instantaneous digital images of fluorescent granules passing the beam of illuminating light.

These measurements resulted in a series of images which where recorded and could be played back as a motion picture. Uncoated granules fluoresced brightly and the form and shape of the granules where clearly depicted, thus demonstrating the feasibility of using fluorescence analysis in preparing enzyme granules. For coated granules the fluorescence was significantly reduced showing that the coating layer reduced the access to the fluorescent marker. Darker areas of the recording thus indicated granules with thicker coatings, thus demonstrating the feasibility of using fluorescence analysis in preparing coated granules.

### Example 4

Fluorescence Analysis of Enzyme Granules with Varying Enzyme Concentration:

An experiment was performed to evaluate if the biologically active could also be the fluorescent marker. Four batches with varying contents of purified protease concentrate were fabricated in a Lödige mixer. The contents of protease were 0 (reference), 1, 4 and 12 KPNU, respectively where KPNU is Kilo NOVO Protease Units, which is determined relatively to a protease standard, and the determination is based on the digestion of a dimethyl casein (DMC) solution by the proteolytic enzyme at standard conditions, i.e. 50° C., pH 8.3, 9 min. reaction time, 3 min. measuring time.

The samples were excitated with an Oriel Xenon Flash Lamp 60000 w/ Oriel attachment 60008 and Oriel Power Supply 68826. Illuminating light was filtered using a band pass filter to produce a beam of light in the region 300-400 nm. The emitted light was detected with a 3-CCD camera from JAI (M-90). The camera was mounted with a Computar 55 Telecentric lens. The signal from the detectors was transferred to a computing unit (ordinary personal computer) equipped with IFC51 frame grabber and Image-Pro Plus version 4.5.

Fluorescent images recorded of the granules showed visually that the average intensity of emitted light increased significantly as a function of increasing concentrations of purified protease. The four batches of granules were subsequently coated with a standard PEG-coating (PEG4000) containing kaolin and titan dioxide. Fluorescent images recorded of the coated granules showed visually that the average intensity of emitted light increased as a function of increasing concentrations of purified protease.

This indicates that the biologically active, in this experiment a protease, may also be the fluorescent marker.

### Example 5

Fluorescence Analysis of Enzyme Granules with Varying Thickness of Coating:

An experiment was performed to investigate whether there is a correlation between the thickness of the coating and the intensity of emitted light. A standard batch of granules containing an enzyme was coated with a PEG-coating (PEG4000) containing kaolin and titan dioxide. The coating was sprayed onto the granules (Huttlin) and samples of granules were withdrawn from the process when 10%, 25% and 50%, respectively, of the coating had been administered. The samples were excitated with an Oriel Xenon Flash Lamp 60000 w/ Oriel attachment 60008 and Oriel Power Supply 68826. Illuminating light was filtered using a band pass filter to produce a beam of light in the region from 300-400 nm. The emitted light was detected with a 3-CCD camera from JAI (M-90). The camera was mounted with a Computar 55 Telecentric lens. The signal from the detectors was transferred to a computing unit (ordinary personal computer) equipped with IFC51 frame grabber and Image-Pro Plus version 4.5.

Fluorescent images recorded of the coated granules showed visually that the average intensity of emitted light decreased for increasing concentrations of coating (i.e. for 10%, 25% and 50% of coating administered).

This indicates that the intensity of fluorescent emitted light can be used to predict the thickness of the coating.

### Example 6

Fluorescence Analysis of Enzyme Granules with Varying Amounts of Enzymatic Active Dust:

An experiment was performed to evaluate if fluorescence analysis could be used to estimate the amount of dust. A priori it is known that coated enzyme granules with scars in the coating lead to problem with enzymatic active dust.

Eight samples with protease active dust values from 29 ng/g to 2420 ng/g of protease per gram of dust were submitted to on-line fluorescence analysis.

The samples were excitated with an Oriel Xenon Flash Lamp 60000 w/ Oriel attachment 60008 and Oriel Power Supply 68826. Illuminating light was filtered using a band pass filter to produce a beam of light in the region from 300-400 nm. The emitted light was detected with a 3-CCD camera from JAI (M-90). The camera was mounted with a Computar 55 Telecentric lens. The signal from the detectors was transferred to a computing unit (ordinary personal computer) equipped with IFC51 frame grabber and Image-Pro Plus version 4.5.

The samples were conveyed to the imaging system in a distance of approximately 10 cm.

Fluorescent images recorded of the granular composition showed visually that the average intensity of emitted light increased for compositions with increasing amounts of protease active dust values.

This indicates that the intensity of fluorescent emitted light can be used to estimate the amount of biologically active dust in granular composition.

1. A method for fluorescence analysis comprising illuminating a granular composition comprising a purified biologically active compound with light capable of fluorescence excitation of a fluorescent marker comprised in the granular composition, detecting light emitted from the fluorescent marker and predicting the amount of fluorescent marker in the granular composition accessible to fluorescence excitation.

2. The method of claim 1, wherein the granular composition is illuminated with a light source producing ultraviolet light having wavelengths between 10-380 nm.

3. The method of claim 2, wherein the ultraviolet light consist of 1-10 discrete monochromatic wavelengths.

4. The method of claim 3, wherein the ultraviolet light consist of one discrete monochromatic wavelength, particularly between 260-280 nm.

5. The method of claim 1, wherein the detecting of light emitted from the fluorescent marker consists of detecting emitted light of 1-10 discrete monochromatic wavelengths, particularly between 185-2600 nm.

6. The method of claim 5, wherein the fluorescent marker is the biologically active compound and the detecting of light emitted from the fluorescent marker consists of detecting emitted light of one discrete monochromatic wavelength, particularly between 300-400 nm.

7. The method of claim 1, wherein the detecting is made with at least one detector capable of converting the emitted light into an electronic signal.

8. The method of claim 7, wherein the detecting is made with a CCD or an ICCD camera capable of converting the emitted light into a digital two-dimensional image.

9. The method of claim 8, wherein the detecting is made with at least two CCD or ICCD cameras capable of converting the emitted light into a digital two dimensional image.

10. The method of claim 1, wherein the prediction is conducted by comparison of light emitted from the particulate composition with light emitted from a particulate composition with known amounts of fluorescent marker.

11. The method of claim 10, wherein the prediction is made in real time.

12. The method of claim 1, wherein the biologically active compound is selected from bio-catalysts, therapeutic agents, herbicides, pesticides and fungicides.

13. The method of claim 12, wherein the biologically active compound is selected from proteins and peptides.

14. The method of claim 13, wherein the biologically active compound is an enzyme, particularly a selected from hydrolases and oxidoreductases.

15. The method of claim 1, wherein the granular composition further comprises auxiliary granulation agents.

16. The method of claim 15, wherein the auxiliary granulation agents are selected from fiber materials, binders, fillers, liquid agents, enzyme stabilizers, suspension agents, cross linking agents, mediators and/or solvents

17. The method of claim 16, wherein the fluorescent marker is an auxiliary granulation agent and the detecting of light emitted from the fluorescent marker consists of detecting emitted light of one discrete monochromatic wavelength, particularly between 350-500 nm.

18. The method of claim 1, wherein the granules comprises a core wherein the biologically active compound is intimately mixed with auxiliary granulation agents.

19. The method of claim 1, wherein the granules comprise a core particle coated with a layer comprising the biologically active compound and particularly auxiliary granulation agents.

20. The method of claim 1, wherein the granules have an average size between 20-2000 μm, particularly between 100-1000 μm, more particularly between 200-800 μm.

21. The method of claim 1, wherein the granules are coated with a coating agent.

22. A process for preparing granules comprising a purified biologically active compound, a fluorescent marker and optionally auxiliary granulation agents in a granulation apparatus said process comprising the step of performing fluorescence analysis on the fluorescent marker in accordance with claims 1-21 on the granules forming in the granulator.

23. The process of claim 22, wherein the fluorescence analysis is performed on-line and in real time during the granulation process and is repeated more than one time during the granulation process.

24. The process of claim 22, further comprising the step of changing at least one process parameter as a result of the fluorescence analysis.

25. The process of claim 24, wherein process parameter is selected from supply of biologically active compound, supply of auxiliary granulation agent, supply of coating agent, supply of gas, temperature, pressure, pH and mechanical force conferred to the granulation material.

26. The process of claim 22-25, further characterized by being a coating process wherein granules comprising a biologically active compound and optionally auxiliary granulation agents are coated with a coating agent and the parameter is supply of coating agent to the granulation apparatus.

27. The process of claim 22, wherein the fluorescence analysis is performed at-line and in real time after the granulation process and repeated more than one time.

28. A granulation or coating apparatus comprising
(a) a granulation or coating device comprising at least one chamber for processing material into granules or coated granules,
(b) an optical arrangement for performing fluorescence analysis comprising a light source for illumination of granules being processed, at least one detector capable of detecting light emitted from the granules being processed, means for projecting illuminating light onto a portion of the granules being processed, means for projecting light emitted from illuminated granules to the detector and at least one filtering device for filtering light.

29. The apparatus of claim 28, wherein the granulating or coating device is selected from a fluid bed granulator or coater, high shear mixer granulator, a spray dryer, a spray cooler, an extruder.

30. The apparatus of claim 28, wherein the light source is selected from a glow lamp, a xenon lamp or a stroboscope lamp, particularly capable of delivering light having wavelengths between 10-700 nm.

32. The apparatus of claim 28, wherein the detector is a camera, particuarly selected from a line-scan camera, a CCD or an ICCD camera.

33. The apparatus of claim 28, wherein the means for projecting illuminating light onto the granules being processed and projecting emitted light from said granules to the detector is selected from one ore more of fiber optics, mirrors, lenses and beam splitters.

34. The apparatus of claim 28, wherein the filtering device is positioned so than only the emitted light is filtered and so that the emitted light must pass the filter before reaching the detector.

35. The apparatus of claim 34, wherein at least two filtering devices are positioned so that both the illuminating light and the emitted light is filtered.

36. The apparatus of claim 28, wherein the optical arrangement comprises a stroboscope light source, 2 CCD camera detectors, one band pass filter for filtering illumination light, two band pass filters for filtering emitted light, a lens, a two dichroic mirror beam splitters.

37. The apparatus of claim 28, wherein the portion of granules being processed is present in the chamber.

38. The apparatus of claim 28, further comprising means for providing a purge stream of granules from the chamber and wherein the optical arrangement is positioned to allow fluorescence analysis of granules in the purge stream.

39. The apparatus of claim 38, wherein the means for providing a purge stream includes means for forming a single layer of granules.

40. The apparatus of claim 39, wherein the means for forming a single layer of granules comprise a tilted vibrating surface.

41. The apparatus of claim 40, wherein the optical arrangement is positioned so that the fluorescence analysis is performed on a single layer of granules after falling over one or more edges of the vibrating surface.

42. The apparatus of claim 28, further comprising one or more elements selected form computing units and control units

43. Use of fluorescence analysis on granules comprising a purified biologically active compound.

44. A method according to claim comprising the steps of:
a) providing a calibration model by illuminating a granular composition comprising a purified biologically active compound having a known quality parameter with light capable of fluorescence excitation of a fluorescent marker comprised in the granular composition, recording one or more images of the light emitted from the granular composition of a known quality and subjecting recorded images to data processing, particularly in the form of partial least squares data processing, to form a calibration model,
b) illuminating a unknown granular composition comprising a purified biologically active compound with light capable of fluorescence excitation of a fluorescent marker comprised in the granular composition, recording at least one image of the light emitted from the unknown granular composition,
c) comparing at least one image of the unknown granular composition with the calibration model and
d) estimating the quality parameter of the unknown granular composition.
